(19) United States
(12) Patent Application Publication
(10) Pub. No.: US 2022/0119857 A1
(43) Pub. Date: Apr. 21, 2022

(54) Use of Muramic Acid as a Biomarker for Gastrointestinal Peptidoglycan Hydrolysis

(71) Applicant: Novozymes A/S, Bagsvaerd (DK)

(72) Inventors: Mikkel Klausen, Copenhagen (DK); Carsten Oestergaard Frederiksen, Kalundborg (DK)

(73) Assignee: Novozymes A/S, Bagsvaerd (DK)

(21) Appl. No.: 17/434,740

(22) PCT Filed: Mar. 20, 2020
(86) PCT No.: PCT/EP2020/057780

§ 371 Date: Aug. 28, 2021

(30) Foreign Application Priority Data
Mar. 22, 2019 (EP) 19164624.9

(51) Int. Cl. C12Q 1/40

(52) U.S. Cl. CPC C12Q 1/40; G01N 30/7233; G01N 2333/4722

(57) ABSTRACT

Muramic acid measurements in acid hydrolysed digesta samples are used to measure the activity of peptidoglycan hydrolyzing enzyme, as illustrated by the use of a muramidase, as determined by the degree of peptidoglycan hydrolysis, in the gastrointestinal tract of animals fed supplements with the muramidase.

### FIELD OF THE INVENTION

The present invention relates to methods of determining muramidase activity by means of peptidoglycan hydrolysis in the gastro-intestinal tract of an animal.

### BACKGROUND OF THE INVENTION

Peptidoglycan (PGN) is a polymer in bacterial cell walls that provides rigidity and shape to the cells, whether spherical, rods, spiral or filamentous. PGN is sometimes called the exoskeleton of bacteria for this reason. The polymeric structure of PGN is exclusive to bacterial cells, and is absent in all other organisms. PGN is a heteropolymer consisting of a sugar backbone with alternating N-acetylglycosamine (NAG) and N-acetylmuramic acid (NAM) components. PGN hydrolases are defined by their catalytic specificities. Two classes of these enzymes function to digest the PGN glycan backbone: N-acetylmuramidases cleave PGN between the NAG-NAM bond upstream of NAM and N-acetylglucosaminidases cleave the NAM-NAG bond. N-acetylmuramyl-L-alanine amidases cleave between NAM and the first alanine of the peptide chain. Catalysis by N-acetylmuramyl-L-alanine amidases separate the PGN sugar backbones from the stem peptide. Lytic transglycosylases cleave between the N-acetylmuramic acid and N-acetylglucosamine sugar chains. Further digestion of the stem peptide requires cleavage between stem amino acids by several carboxy- and endopeptidases. Glycosidases, amidases, and endopeptidases are also required to provide bacteria the capacity to fully degrade PGN and recycle cell wall glycans and amino acids.

Muramic acid (MurA) is present exclusively in nature in cell walls of bacteria. It has been used as a biomarker for estimating bacterial biomass various complex biological samples such as dust, soil and body fluids. Balkwill et al. (Equivalence of microbial biomass measures based on membrane lipid and cell-wall components, adenosine-triphosphate, and direct counts in subsurface aquifer sediments. Microbial Ecol 16:73-84, 1988) compared four methods of microbial biomass determination in subsurface sediment, and concluded that biomass measured by MurA, is equivalent to lipid, adenosine triphosphate (ATP), and direct counting. Bak and Larsson (New and simple procedure for the determination of muramic acid in chemically complex environments by gas chromatography-ion trap tandem mass spectrometry J Chromatogr B Biomed Sci Appl 738(1):57-65, 2000) measured muramic acid in bacteria, house dust and urine.

The widely accepted assay for analyzing muramic acid comprises aldononitrile acetate derivatization.

Muramic acid measurements are described for estimating bacterial abundances soil and dust but has not been applied to measure effects of digestive muramidases.

The use of muramidases (lysozymes) as digestive aids has been reported to improve animal performance in several independent studies. Some muramidases are believed to function through antimicrobial activity whereas others without antimicrobial potency are thought to hydrolyse bacterial cell debris in the gut.

Protein digestibility can be used to measure effect of proteases. Phosphate release can be used to measure effects of phytases. A method to measure the effect of the muramidase on its substrate in the digestive tract has however not been reported.

### SUMMARY OF THE INVENTION

Herein is described a method in which muramic acid measurements in acid hydrolysed digesta samples and acid hydrolysed digesta sample extracts can be used to measure effect of muramidase used as digestive aids. The invention provides for an assay for measuring peptidoglycan hydrolysis by a muramidase or by a peptidoglycan hydrolyzing enzyme in a biological sample, including an assay for measuring peptidoglycan hydrolysis in a biological sample comprising the use of a muramidase or by a peptidoglycan hydrolyzing enzyme and applying said muramidase or by a peptidoglycan hydrolyzing enzyme. The invention provides a method to measure peptidoglycan degree of hydrolysis in the gastrointestinal tract of broilers supplemented with an enzyme, typically a muramidase. The invention further provides a method of determining the relative amount of soluble peptidoglycan in the gastrointestinal tract comprising determining the amount of soluble muramic acid relative to the total muramic acid in the sample. A general aspect of the invention is directed to an assay for measuring peptidoglycan hydrolysis by an enzyme, typically a muramidase, in a biological sample.

The invention is furthermore directed to a method of determining the peptidoglycan hydrolysis activity of an enzyme, typically a muramidase, in a biological sample comprising measuring the amount of soluble peptidoglycan in sample comprising said muramidase.

Alternatively stated, the method of the invention is directed to determining the in-vivo peptidoglycan hydrolysis activity of a muramidase comprising measuring the amount of soluble peptidoglycan in a biological a sample taken from an animal.

An aspect of the invention is directed to a method of quantifying soluble peptidoglycan in the gastrointestinal tract in absolute or relative terms. This aspect comprises determining the amount of soluble muramic acid relative to the total muramic acid in the sample. Similarly, the invention relates to determining the ratio of soluble peptidoglycan to insoluble peptidoglycan in any portion of an animal's digestive tract comprising determining the amount of soluble muramic acid relative to the total muramic acid in the sample.

A further aspect of the invention relates to determining the relative peptidoglycan hydrolysis activity of a muramidase in different parts of a digestive tract comprising measuring the amount of soluble peptidoglycan in at least two different parts of a digestive tract.

The method of the invention may be defined, in a further aspect, as a method of determining the relative in-vivo activity of two or more muramidases, said activity determined by the relative amount of soluble peptidoglycan is one or more parts of the digestive tract, comprising determining the amount of soluble muramic acid relative to the total muramic acid in the sample.

The method of the invention may be alternatively defined as a method of determining the relative in-vivo activity of two or more doses of one or more muramidases, said activity determined by the relative amount of soluble peptidoglycan is one or more parts of the digestive tract, comprising determining the amount of soluble muramic acid relative to the total muramic acid in the sample.

### BRIEF DESCRIPTION OF FIGURES

For the Figures, data stems from two independent animal trials. FIGS. 1, 2 and 3 stem from trial A and FIGS. 4 and 5 stem from trial B.

FIG. 1 shows the increase in the percent of soluble peptidoglycan in the jejunum (the remainder being insoluble peptidoglycan) upon exogenous delivery of a test muramidase (Balancius™) compared to the Control (Balancius™ is commercially available from DSM as a microbial muramidase that supports digestion, gastrointestinal functionality and improves animal performance delivered by means of the animal feed. The GH25 polypeptide is described in WO 2017/001703). Jejunum samples were analysed from an in vivo trial where a control group without supplemented muramidase and 3 groups with three different concentrations of a muramidase were supplemented to broiler chickens. From this example, the amount of soluble peptidoglycan is shown to significantly increase in the jejunum compared to the control. From the method of the invention, the amount of soluble peptidoglycan be determined for the test muramidase. The amount of soluble peptidoglycan in the jejunum is determined to be between approximately 60% to 80%, depending on the dose; or an increase of about 50% to 70% compared to the control. Accordingly, the method of the invention shows a 5-fold to 7-fold increase in the percentage of soluble peptidoglycan in the jejunum according to the method of the invention. The Figure shows that muramic acid measurements of the invention are an efficient and effective tool to measure peptidoglycan degree of hydrolysis in biological samples such as intestinal samples. Bars with different letter labels are significantly different whereas bars with identical letter label are not.

FIG. 2 shows the increase in the percent of soluble peptidoglycan in the ileum (the remainder being insoluble peptidoglycan) upon exogenous delivery of a test muramidase (delivered by means of the animal feed) compared to the Control. Ileum samples were analysed from an in vivo trial where a control group without supplemented muramidase and 3 groups with three different concentrations of a muramidase were supplemented to broiler chickens. From this example, the amount of soluble peptidoglycan is shown to significantly increase in the ileum compared to the control. Also, the absolute relative activity of the muramidase at different dosages can be determined. In the present instance in the ileum, the increase in soluble peptidoglycan is dose dependent. From the method of the invention, the amount of soluble peptidoglycan can be determined for the test muramidase. The amount of soluble peptidoglycan in the ileum is determined to be between approximately 40% to 65%, depending on the dose; or an increase of about 20% to 45% compared to the control. Accordingly, the method of the invention shows a 2-fold to 4.5-fold increase in the percentage of soluble peptidoglycan in the ileum according to the method of the invention. The Figure shows that muramic acid measurements of the invention are an efficient and effective tool to measure peptidoglycan degree of hydrolysis in biological samples such as intestinal samples. Bars with different letter labels are significantly different whereas bars with identical letter label are not.

FIG. 3 shows the increase in the percent of soluble peptidoglycan in the caecum (the remainder being insoluble peptidoglycan) upon exogenous delivery of a test muramidase (delivered by means of the animal feed) compared to the Control. Caecum samples were analysed from an in vivo trial where a control group without supplemented muramidase and 3 groups with three different concentrations of a muramidase were supplemented to broiler chickens. From this example, the amount of soluble peptidoglycan is shown to significantly increase in the caecum compared to the control. Also, the absolute relative activity of the muramidase at different dosages can be determined. From the method of the invention, the amount of soluble peptidoglycan be determined for the test muramidase. The amount of soluble peptidoglycan in the caecum is determined to be between approximately 10% to 17.5%, depending on the dose; or an increase of about 5% to 12.5% compared to the control. Accordingly, the method of the invention shows a 2-fold to 3.5-fold increase in the percentage of soluble peptidoglycan in the caecum according to the method of the invention. The Figure shows that muramic acid measurements of the invention are an efficient and effective tool to measure peptidoglycan degree of hydrolysis in biological samples such as intestinal samples. Bars with different letter labels are significantly different whereas bars with identical letter label are not.

FIG. 4 shows the increase in the percent of soluble peptidoglycan in the jejunum (the remainder being insoluble peptidoglycan) upon exogenous delivery of a test muramidase (delivered by means of the animal feed) compared to the Control. Jejunum samples were analysed from an in vivo trial where a control group without supplemented muramidase where compare to a muramidase supplemented group of broiler chickens. From this example, the amount of soluble peptidoglycan is shown to increase slightly more than 2-fold in the jejunum upon administration of the test muramidase in feed. The Figure shows that muramic acid measurements of the invention are an efficient and effective tool to measure peptidoglycan degree of hydrolysis in biological samples such as intestinal samples. Bars with different letter labels are significantly different whereas bars with identical letter label are not.

FIG. 5 shows the increase in the percent of soluble peptidoglycan in the excreta (the remainder being insoluble peptidoglycan) upon exogenous delivery of a test muramidase (delivered by means of the animal feed) compared to the Control. Excreta samples were analysed from an in vivo trial where a control group without supplemented muramidase where compare to a muramidase supplemented group of broiler chickens. From this example, the amount of soluble peptidoglycan is shown to increase slightly less than 2-fold in the excreta upon administration of the test muramidase in feed. The Figure shows that muramic acid measurements of the invention are an efficient and effective tool to measure peptidoglycan degree of hydrolysis in biological samples such as intestinal samples. Bars with different letter labels are significantly different whereas bars with identical letter label are not.

### DETAILED DESCRIPTION OF THE INVENTION

The measurement of muramic acid in biological samples has until know been used as a biomarker of bacterial mass in complex samples, because it is uniquely found in bacterial cells walls. In the present invention muramic acid measurements are used as a tool to measure peptidoglycan degree of hydrolysis in biological samples such as intestinal samples.

A first aspect of the invention is directed to an assay for measuring peptidoglycan hydrolysis by a muramidase or by a peptidoglycan hydrolyzing enzyme in a biological sample such as an assay for measuring peptidoglycan hydrolysis by a muramidase in a biological sample. The muramidase is typically an exogenous muramidase, such as a muramidase added to the animal feed or water or otherwise administered to the animal. The muramidase is preferably a digestive enzyme. The peptidoglycan hydrolyzing enzyme may be a peptidoglycan hydrolase selected from the group consisting of a muramidase or N-acetylmuramidase, a N-acetylglucosaminidase, a N-acetylmuramyl-L-alanine amidases, a lytic transglycosylase, a carboxypeptidase, an endopeptidase, a glycosidases, or an amidase. Muramidases (EC 3.2.1.17), also known as N-acetylmuramidase, hydrolyze PGN between the N-acetylmuramic acid and N-acetylglucosamine and are a preferred embodiment of the invention. Further preferred embodiments may be slected from the group consisting of N-acetylglucosaminidases, a N-acetylmuramyl-L-alanine amidases, and lytic transglycosylases.

A further aspect of the invention is directed to a method of determining the peptidoglycan hydrolysis activity of a muramidase in a biological sample comprising measuring the amount of soluble peptidoglycan in said sample comprising said muramidase. An alternate aspect of the invention is directed to method of determining the relative amount of soluble peptidoglycan in the gastrointestinal tract comprising determining the amount of soluble muramic acid relative to the total muramic acid in the sample.

The activity of a muramidase is important in the selection process of enzymes for use in for commercial purposes. Accordingly, testing the in-vivo activity of an enzyme is highly relevant. According to the present invention, this is done by measuring the amount of soluble peptidoglycan in a sample taken from the animal. An aspect of the invention is directed to a method of determining the in-vivo peptidoglycan hydrolysis activity of a muramidase comprising measuring the amount of soluble peptidoglycan in a biological sample taken from an animal. Given the muramidase is typically exogenously added, the method of determining the in-vivo peptidoglycan hydrolysis activity of a muramidase comprising measuring the amount of soluble peptidoglycan in a biological sample taken from an animal is typically also method of quantifying soluble peptidoglycan in the gastrointestinal tract.

The method of the invention can measure the absolute or relative amount of soluble peptidoglycan in a biological sample as a measure of the muramidase activity. Accordingly, the invention is further directed to a method of determining the ratio of soluble peptidoglycan to insoluble peptidoglycan in any portion of an animal's digestive tract. The amount or ratio of soluble peptidoglycan to insoluble peptidoglycan may be measured by measuring the overall amount in the digestive tract, in any part of the digestive tract for absolute readings in any one part of the tract such as the jejunum, the ileum, the caecum, and/or the crop, or in more than one part of the digestive for relative measurements of soluble peptidoglycan, or relative ratios of soluble peptidoglycan to insoluble peptidoglycan in two or more parts of digestive tract of the jejunum, the ileum, the caecum, and/or the crop. Alternatively, or additionally, the ratio of soluble peptidoglycan to insoluble peptidoglycan is measured in the excretion. An embodiment relates to a method of determining the relative peptidoglycan hydrolysis activity of a muramidase in different parts of a digestive tract comprising measuring the amount of soluble peptidoglycan in at least two different parts of a digestive tract.

A related aspect of the invention is directed to a method of determining the relative in-vivo activity of two or more muramidases, said activity determined by the relative amount of soluble peptidoglycan is one or more parts of the digestive tract, comprising determining the amount of soluble muramic acid relative to the total muramic acid in the sample. In a suitable embodiment, comparing the relative activity of two or more muramidases comprises comparing the overall amount of soluble peptidoglycan in the digestive tract and/or in the excrement of an animal; or by comparing the ratio of soluble peptidoglycan to insoluble peptidoglycan in the digestive tract and/or in the excrement of an animal; or by comparing the overall amount of soluble peptidoglycan in any of the jejunum, the ileum, the caecum, the crop, and/or the excrement of an animal; or by comparing the ratio of soluble peptidoglycan to insoluble peptidoglycan in any of the jejunum, the ileum, the caecum, the crop, and/or the excrement of an animal. An additional aspect of the invention is directed to a method of determining the relative in-vivo activity of two or more doses of one or more muramidases, said activity determined by the relative amount of soluble peptidoglycan is one or more parts of the digestive tract, comprising determining the amount of soluble muramic acid relative to the total muramic acid in the sample. In a suitable embodiment, comparing the relative activity of two or more doses of one or more muramidases comprises comparing the overall amount of soluble peptidoglycan in the digestive tract and/or in the excrement of an animal; or by comparing the ratio of soluble peptidoglycan to insoluble peptidoglycan in the digestive tract and/or in the excrement of an animal; or by comparing the overall amount of soluble peptidoglycan in any of the jejunum, the ileum, the caecum, the crop, and/or the excrement of an animal; or by comparing the ratio of soluble peptidoglycan to insoluble peptidoglycan in any of the jejunum, the ileum, the caecum, the crop, and/or the excrement of an animal.

According to the invention, the method typically comprises an initial step wherein water-soluble peptidoglycan is separated from insoluble peptidoglycan. Subsequently the total amount of muramic acid is determined in each sample as well as the amount of muramic acid in the soluble phase. The percentage of soluble muramic acid relative to the total muramic acid in the sample is determined and may be used as a measurement of peptidoglycan degree of hydrolysis of peptidoglycan.

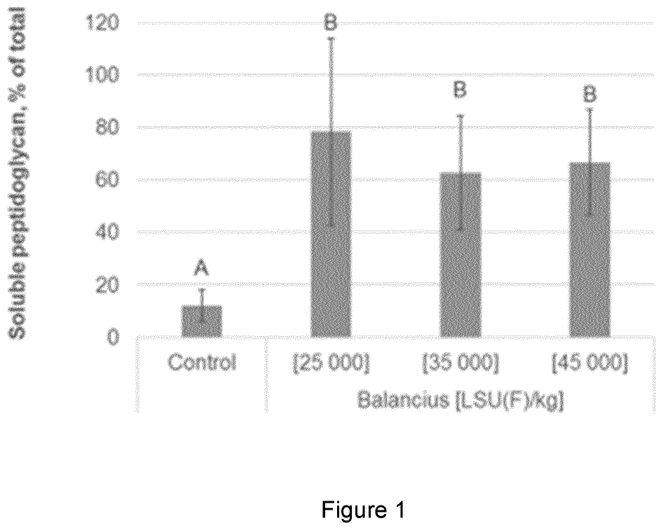

As illustrated by FIG. 1, one aspect of the invention is directed to determining the increase in soluble peptidoglycan in the jejunum (the remainder being insoluble peptidoglycan) by exogenous delivery, such as by adding a muramidase to an animal feed, to an animal. The animal may be broiler chickens. According to the method of the invention, the amount of soluble peptidoglycan may be determined in the jejunum for a test muramidase and compared to a control. According to the invention, the relative activity of test muramidases may be compared by determining the increase in soluble peptidoglycan in the jejunum by exogenous delivery, such as by adding a muramidase to an animal feed, to an animal.

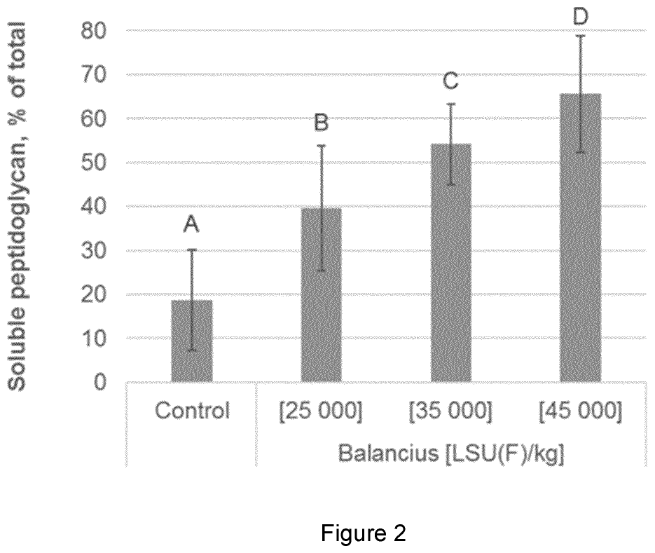

As illustrated by FIG. 2 one aspect of the invention is directed to determining the increase in soluble peptidoglycan in the ileum (the remainder being insoluble peptidoglycan) by exogenous delivery, such as by adding a muramidase to an animal feed, to an animal. The animal may be broiler chickens. According to the invention, the relative activity of test muramidases may be compared by determining the increase in soluble peptidoglycan in the ileum by exogenous delivery, such as by adding a muramidase to an animal feed, to an animal. As further seen by FIG. 2, the relative activity of test muramidases at varying doses may be determined. As shown by FIG. 2, the increase in soluble peptidoglycan in the ileum may be dose dependent. According to the invention, the method may comprise determining the relative activity of test muramidases at varying doses by determining the relative amount of soluble peptidoglycan in the ileum of an animal, such as a broiler chicken.

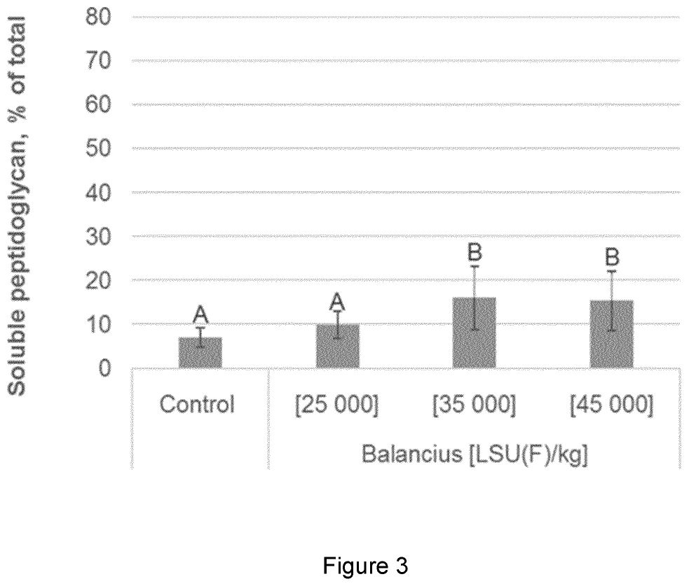

As illustrated by FIG. 3 one aspect of the invention is directed to determining the increase in soluble peptidoglycan in the caecum (the remainder being insoluble peptidoglycan) by exogenous delivery, such as by adding a muramidase to an animal feed, to an animal. The animal may be broiler chickens. According to the invention, the relative activity of test muramidases may be compared by determining the increase in soluble peptidoglycan in the caecum by exogenous delivery, such as by adding a muramidase to an animal feed, to an animal. As further seen by FIG. 3, the relative activity of test muramidases at varying doses may be determined. As shown by FIG. 3, the increase in soluble peptidoglycan in the caecum may be dose dependent. According to the invention, the method may comprise determining the relative activity of test muramidases at varying doses by determining the relative amount of soluble peptidoglycan in the caecum of an animal, such as a broiler chicken.

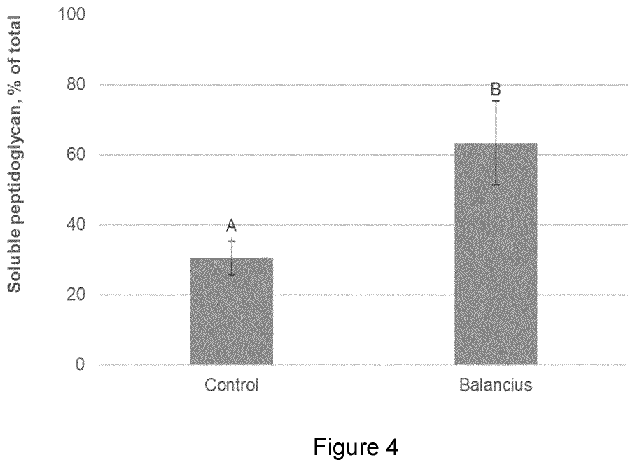

As illustrated by FIG. 4 one aspect of the invention is directed to determining the increase in soluble peptidoglycan in the jejunum (the remainder being insoluble peptidoglycan) by exogenous delivery, such as by adding a muramidase to an animal feed, to an animal. The animal may be broiler chickens. According to the invention, the relative activity of test muramidases may be compared by determining the increase in soluble peptidoglycan in the jejunum by exogenous delivery, such as by adding a muramidase to an animal feed, to an animal. As further seen by FIG. 4, the relative activity of test muramidases at varying doses may be determined. As shown by FIG. 4, the increase in soluble peptidoglycan in the jejunum may be dose dependent. According to the invention, the method may comprise determining the relative activity of test muramidases at varying doses by determining the relative amount of soluble peptidoglycan in the jejunum of an animal, such as a broiler chicken.

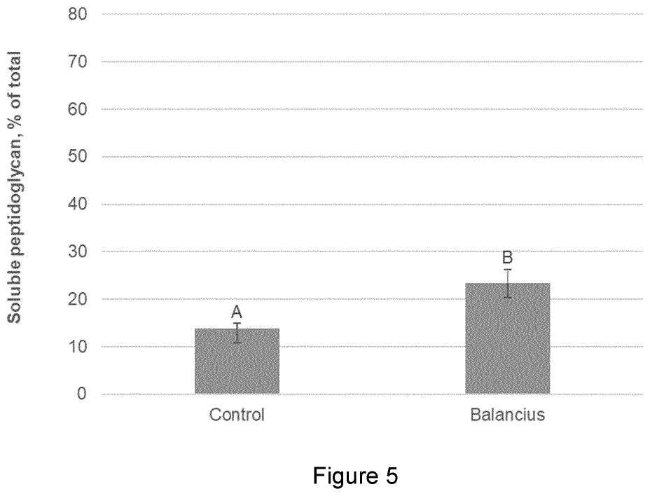

As illustrated by FIG. 5 one aspect of the invention is directed to determining the increase in soluble peptidoglycan in the excreta (the remainder being insoluble peptidoglycan) by exogenous delivery, such as by adding a muramidase to an animal feed, to an animal. The animal may be broiler chickens. According to the invention, the relative activity of test muramidases may be compared by determining the increase in soluble peptidoglycan in the excreta by exogenous delivery, such as by adding a muramidase to an animal feed, to an animal. As further seen by FIG. 5, the relative activity of test muramidases at varying doses may be determined. As shown by FIG. 5, the increase in soluble peptidoglycan in the jejunum may be dose dependent. According to the invention, the method may comprise determining the relative activity of test muramidases at varying doses by determining the relative amount of soluble peptidoglycan in the excreta of an animal, such as a broiler chicken.

A further aspect of the invention is directed to a muramidase identified by or selected by a method of the invention.

A further aspect of the invention is to use the method to measure muramidase activity in other body samples, food production or diagnostics.

### Embodiments

1. An assay for measuring peptidoglycan hydrolysis by a muramidase or by a peptidoglycan hydrolyzing enzyme in a biological sample.
2. An assay according to embodiment 1 for measuring peptidoglycan hydrolysis by a muramidase in a biological sample.
3. An assay for measuring peptidoglycan hydrolysis comprising the use of a muramidase or by a peptidoglycan hydrolyzing enzyme.
4. An assay for measuring peptidoglycan hydrolysis comprising the use of a muramidase or by a peptidoglycan hydrolyzing enzyme and applying said muramidase or by a peptidoglycan hydrolyzing enzyme to a biological sample.
5. An assay for measuring peptidoglycan hydrolysis in a biological sample comprising the use of a muramidase or by a peptidoglycan hydrolyzing enzyme and applying said muramidase or by a peptidoglycan hydrolyzing enzyme.
6. An assay for measuring peptidoglycan hydrolysis comprising the use of a muramidase or by a peptidoglycan hydrolyzing enzyme and applying said muramidase or by a peptidoglycan hydrolyzing enzyme to a biological sample.
7. An assay according to any of embodiments 1, 2 and 4 to 6 wherein the biological sample is taken from an animal.
8. An assay according to embodiment 7, wherein the biological sample is a taken from the digestive tract of an animal.
9. An assay according to embodiment 7, wherein the biological sample is a taken from the intestinal tract of an animal.
10. A method of determining the peptidoglycan hydrolysis activity of a muramidase in a biological sample comprising measuring the amount of soluble peptidoglycan in a sample comprising said muramidase.
11. A method of determining the in-vivo peptidoglycan hydrolysis activity of a muramidase comprising measuring the amount of soluble peptidoglycan in a biological sample taken from an animal.
12. A method of determining the relative amount of soluble peptidoglycan in the gastrointestinal tract comprising determining the amount of soluble muramic acid relative to the total muramic acid in the sample.
13. A method of determining the ratio of soluble peptidoglycan to insoluble peptidoglycan in any portion of an animal's digestive tract.
14. A method according to embodiment 13, wherein the ratio of soluble peptidoglycan to insoluble peptidoglycan is measured in any one of the jejunum, the ileum, the caecum, and/or the crop.
15. A method according to embodiment 14, wherein the ratio of soluble peptidoglycan to insoluble peptidoglycan is measured in the excretion.
16. A method of determining the relative peptidoglycan hydrolysis activity of a muramidase in different parts of a digestive tract comprising measuring the amount of soluble peptidoglycan in at least two different parts of a digestive tract.
17. A method of determining the relative in-vivo activity of two or more muramidases, said activity determined by the relative amount of soluble peptidoglycan is one or more parts of the digestive tract, comprising determining the amount of soluble muramic acid relative to the total muramic acid in the sample.
18. A method of determining the relative in-vivo activity of two or more doses of one or more muramidases, said activity determined by the relative amount of soluble peptidoglycan is one or more parts of the digestive tract, comprising determining the amount of soluble muramic acid relative to the total muramic acid in the sample.
19. A muramidase identified by a method according to any one of embodiments 10 to 18.

### EXAMPLES

### Example 1

### Method

Preparation of samples: Intestinal samples are collected and frozen as quickly as practically possible. Sample are then freeze dried and grinded to ensure homogeneity 100 mg of each sample is then collected to determine the total amount of muramic acid. Another 100 mg is dissolved in 0.8 mL buffer at pH 6 and incubated 95° C. for 15 min to inactivate enzyme activity. Samples are then extracted for 45 min at 23° C. with shaking after which they are centrifuged at 13000 RPM at 5° C. for 5 min. Supernatant is collected to determine the concentration of soluble muramic acid.

Acidic hydrolysis: The sample is either weighed or pipetted, depending on the sample being solid or an extraction, into a glass vial. The sample is hydrolyzed using a resulting concentration of 5 M hydrochloric acid for 24 hours at 100° C. The hydrolysate is dried in a freeze dryer under vacuum.

### Derivatization and Analysis

The dried hydrolysate is reconstituted in ultrapure water and centrifuged. Derivatization is conducted by mixing 200 μL supernatant or standard, 20 μL 0.1 mg/mL 6-deoxy-D-glucose (internal standard), 20 μL 4M NaOH and 200 μL 0.5M 1-phenyl-3-methyl-5-pyrazolone (PMP) in methanol. The capped vials are incubated at 70° C. for 30 min. The derivatized sample is neutralized by adding 20 μL 4M hydrochloric acid followed by addition of 400 μL methanol. Samples and standards are diluted in 50% methanol/water using the same dilution factor and analysed for muramic acid by UPLC-MS as directed below.

Samples are analysed by reverse phase chromatography using a Waters Acquity UPLC CSH C18 analytical column (2.1×50 mm, 1.7 μm particle size, 130 Å pore size). UPLC analysis was performed using a binary gradient at a flow of 0.5 mL/min using an Acquity UPLC (Waters). Mobile phases consist of ultrapure water with 0.15% formic acid (A) and acetonitrile (ACN) with 0.15% formic acid (B). A linear gradient was started at 90% solvent A, which was changed within 10 minutes to 78% solvent A. The composition was then changed to 95% B within 0.1 min and after cleaning the column with 95% solvent B for 1 minute, the column was equilibrated for 2 minutes at the initial composition. Injection volume was 4 μL, and column temperature was set at 60° C. Detection was carried out using a Waters Xevo TQ-S micro triple quadrupole mass spectrometer operated in positive electrospray ionization mode.

Optimal conditions were found at a capillary voltage of 3 kV and a cone voltage of 20V. The source and desolvation temperature were 150 and 500° C., respectively. The cone gas flow and desolvation gas flow were 20 and 600 L/hour, respectively. Bis-PMP-muramic (M+2H)²⁺=291.3 Da and bis-PMP-6-deoxy-D-glucose (M+H)⁺=495.2 Da were chosen as the detected ions in the selected ion recording (SIR) mode.

### Results

### Intestinal Sample Set A

Jejunum, Ileum and Caecum samples were analysed from an in vivo trial where a control group without supplemented muramidase and 3 groups with three different concentrations of a muramidase were supplemented to broiler chickens. 18 unique samples from different animals were analysed in each group and compared using Turkey-Kramer HSD, p<0.05 statistical test in SAS JMP.

### Intestinal Sample Set B

Jejunum and excreta samples were analysed from an in vivo trial where a control group without supplemented muramidase where compare to a muramidase supplemented group of broiler chickens. 48 unique samples from different animals were analyzed in each group and compared using Turkey-Kramer HSD, p<0.05 statistical test in SAS JMP.

1. An assay for measuring peptidoglycan hydrolysis by a muramidase or by a peptidoglycan hydrolyzing enzyme in a biological sample.

2. An assay according to claim 1 for measuring peptidoglycan hydrolysis by a muramidase in a biological sample.

3. A method of determining the peptidoglycan hydrolysis activity of a muramidase in a biological sample comprising measuring the amount of soluble peptidoglycan in a sample comprising said muramidase.

4-12. (canceled)
